(19) United States
(12) Patent Application Publication
(10) Pub. No.: US 2005/0220896 A1
(43) Pub. Date: Oct. 6, 2005

(54) Antimicrobial Articles and Compositions Made from Thermoplastic Elastomers

(75) Inventor: Aldo A. Laghi, Clearwater, FL (US)

Correspondence Address: CARLTON FIELDS, PA, P.O. BOX 3239, TAMPA, FL, 33601-3239, US

(73) Assignee: ALPS SOUTH CORPORATION, St. Petersburg, FL (US)

(21) Appl. No.: 10/907,472

(22) Filed: Apr. 1, 2005

Related U.S. Application Data
(63) Continuation-in-part of application No. 10/817,612, filed on Apr. 2, 2004.

(51) Int. Cl. A61K 33/38

(52) U.S. Cl. 424/618; 523/122

(57) ABSTRACT

A composition for skin contact applications and for treating wounds having antimicrobial agents dispersed in a thermoplastic elastomer. The antimicrobial agents migrate to the surface of the thermoplastic elastomer to keep the wound and the skin free of infection.

### PRIORITY CLAIM

This application is a continuation in part of U.S. patent application Ser. No. 10/817,612 filed Apr. 2, 2004 and entitled “Precipitation of additives in over-saturated triblock copolymer elastomers” the specification of which is incorporated herein by reference.

### FIELD OF INVENTION

This invention relates to an antimicrobial thermoplastic elastomer for skin contact applications, pressure ulcers and wound management.

### BACKGROUND

Diabetics and other patients with impaired vascularity are easily subjected to ischemia and consequently to pressure ulcers. Ischemia is localized tissue anemia due to obstruction of the flow of arterial blood. Ischemia occurs when too much pressure is applied to one area for a prolonged period of time. This pressure is usually from a bony prominence on one side and a hard surface on the other side. The soft tissue between these bony prominences and the hard surface is subjected to abnormal pressure. The ischemia, if prolonged, eventually leads to necrosis of the tissues.

The arterial pressure in the capillaries is about 30 mmHg, and the venous pressure in the capillaries is about 12 mmHg. Prolonged external pressure higher that 12 mmHg can result in ischemia and consequently tissue necrosis. Also frictional and shear (tangential) forces on the skin contribute to tissue necrosis. Venous ulcerations are the most common type of ulcer that affects the lower extremities. Normal veins have valves that prevent the back flow of blood; if these veins become incompetent, the back flow of venous blood causes venous congestion.

Hemoglobin from red blood cells leaks into extra vascular tissue and causes the brown discoloration often observed. Venous ulcers typically appear near the medial malleolus. Treatment of venous ulcers requires: (1) application of antimicrobials to keep the wound site free of infection; (2) retention of moisture to keep the wound in a moist environment; and (3) 40 mmHg compression to control edema.

### SUMMARY OF INVENTION

The present invention is a composition for controlling microbial activity with skin contact applications and for treating wounds including a thermoplastic elastomer of predetermined modulus sufficient to maintain a substantially uniform pressure on a wound and a dispersion of silver-based antimicrobial agents in the thermoplastic elastomer. Application of the thermoplastic elastomer over the wound or the skin permits the migration of the antimicrobial agent from the surface of the elastomer to the skin. The presence of the antimicrobial agent keeps the skin and the wound site free of infection.

The thermoplastic elastomer may be impermeable to water whereby retention of moisture to the wound and skin is achieved. The soft nature of the elastomer enables controlled compression of the wound to prevent ischemia. The soft nature of the elastomer also minimizes frictional and shear forces on the skin. As a consequence tissue necrosis is virtually eliminated. The silver-based antimicrobial agent may include silver zeolite, silver zirconium phosphate, silver nitrate, silver thiosulfate, silver sulphadiazine, and silver fusidate. Other classes of silver compounds may be used as well such as: silver acetate, silver bromide, silver carbonate, silver chlorate, silver chloride, silver citrate, silver fluoride, silver iodate, silver lactate, silver nitrate, silver nitrite, silver perchlorate or silver sulfide

It is also anticipated that the silver-based antimicrobial agent may be used in conjunction with other antimicrobial compounds.

The preferred thermoplastic elastomer is created by mixing together plasticizing oil, triblock copolymer and an antimicrobial agent to form a mixture which is melted then cooled into the thermoplastic elastomer. Alternatively, the antimicrobial agents may be added to the mixture after it is melted or during the cooling process. During cooling the thermoplastic elastomer may be formed into any number of articles including, but not limited to, prosthetic liners, prosthetic sleeves, external breast prostheses, breast enhancement bladders, wound dressing sheets, wound dressing pads, socks, gloves, malleolus pads, metatarsal pads, shoe insoles, urinary catheters, vascular catheters and balloons for medical catheteters both vascular as well as urinary. The polymer may include, but is not limited to triblock copolymer of the type: styrene-ethylene-ethylene-propylene-styrene, styrene-ethylene-butylene-styrene, and styrene-ethylene-propylene-styrene.

### BRIEF DESCRIPTION OF THE DRAWINGS

For a fuller understanding of the invention, reference should be made to the following detailed description, taken in connection with the accompanying drawings, in which:

FIG. 1A is a diagrammatic view of an embodiment of the invention.

FIG. 1B is a diagrammatic view of an alternate embodiment of the invention.

FIG. 1C is a diagrammatic view of an alternate embodiment of the invention.

FIG. 2A is a side-elevated, partially sectional diagrammatic view of the elastomer surface in contact with an epidermal surface where no antimicrobial particles are used.

FIG. 2B is a side-elevated, partially sectional diagrammatic view of the elastomer surface in contact with an epidermal surface showing how the antimicrobial particles of the present invention interfere with microbial activity.

FIG. 3 is an elevated, magnified, partially sectional view of an embodiment of the invention as a sleeve around a wounded knee.

FIG. 4 is an elevated, magnified, partially sectional view of an embodiment of the invention as a prosthetic liner on a residual limb of an amputee.

### DETAILED DESCRIPTION

The subject of this invention is a thermoplastic elastomer which contains antimicrobial agents, provides a moisture barrier by being impermeable to water, and in its softer formulations, distributes pressure evenly on the skin surface and virtually eliminates shear forces on the skin.

The novel elastomer composition is formed by mixing together plasticizing oil, pre-selected additives of which at least one exhibits antimicrobial characteristics (herein “antimicrobial agent”), and a polymer to form a mixture. The mixture is heated until it becomes molten and the molten mixture is charged into a mold for producing useful items.

The antimicrobial agent is kept in suspension when the mixture is in its molten state. An elastomer is formed when the molten mixture cools and solidifies. The antimicrobial agent is present on the surface of the elastomer where it comes in contact with a user's skin and/or wound.

The plasticizing oil may be heated prior to mixing the antimicrobial agent and polymer therewith, but such heating is not strictly necessary. An extruder, a molding machine, or other similar heated vessel is used to accomplish the above-mentioned melting of the mixture so that the antimicrobial agent becomes suspended or dissolved in the molten mixture.

Once the elastomer cools and solidifies, the antimicrobial agent in part remains in the bulk of the elastomer, in part is present on the surface of the elastomer. Such an elastomer can be molded or extruded or thermoformed into various shapes and items such as prosthetic liners and sleeves, external breast prostheses, seals for CPAP (Continuous Positive Air Pressure) masks, wound dressing sheets and pads, socks for diabetic feet, malleolus pads, metatarsal pads, shoe insoles, catheters and balloons for catheters.

As used herein, the term “elastomers” refers to materials having attributes dissimilar from gels. One important attribute of a gel is that its physical state is normally neither a liquid nor a solid. Gels do not seek to fill a container and do not necessarily have a level surface. Gels keep their own shape subjected to gravity. Gels self-heal when cut, have substantially no resistance to traction, and have substantially no elongation.

Examples of true gels are the gels used in food gelatins, wound care, and lubricants. In the present disclosure, triblock polymers, when mixed with plasticizing oils, produce elastomers that exhibit good properties, including elongation, tear and tensile strength characteristics.

Typical modulus of elasticity, measured at 300% elongation, for the softer formulations of this elastomer are in the range of about 5 psi to 50 psi. exhibiting elongations at break in the range of 600% to 2500%. The modulus of elasticity for a material is the ratio between the force required to stretch the material to a given length (represented as a percentage of its original length) and the cross section of the material prior to stretching. For example, the force required for 0% elongation is 0, values increase in substantially linear relation as force is applied to any given material. Accordingly, the higher the modulus the stiffer the material. The modulus is inversely proportion to the amount of plasticizing oil in the composition of the elastomeric gel. Table 1 provides the modulus for various concentrations of plasticizing oil (as measured by parts oil by weight per 100 parts of polymer) in the novel composition.

TABLE 1

| PHR oil | % 300 Modulus |
|---|---|
| (parts oil by weight per 100 parts of | (tensile modulus PSI measured at |
| polymer) | 300% elongation) |
| 300 | 55 |
| 400 | 44 |
| 500 | 35 |
| 600 | 24 |
| 700 | 15 |
| 800 | 8 |

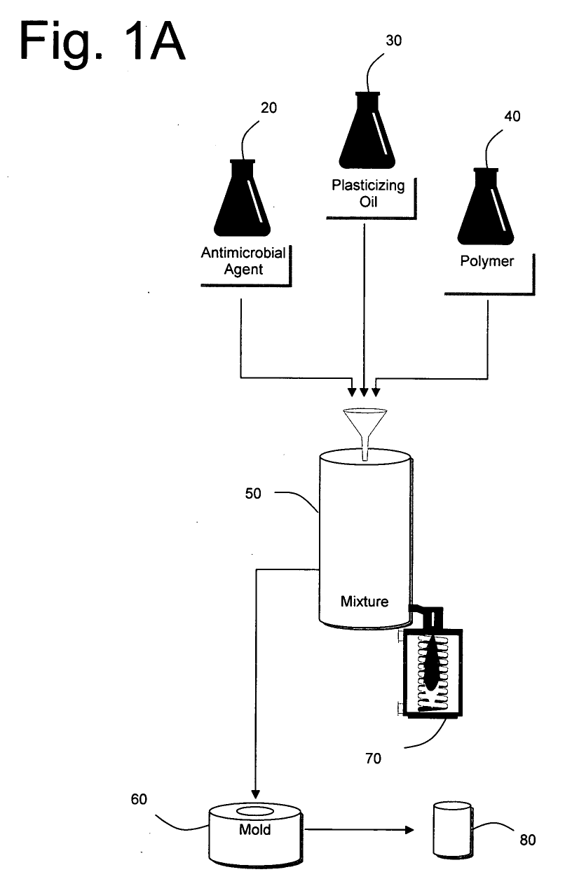

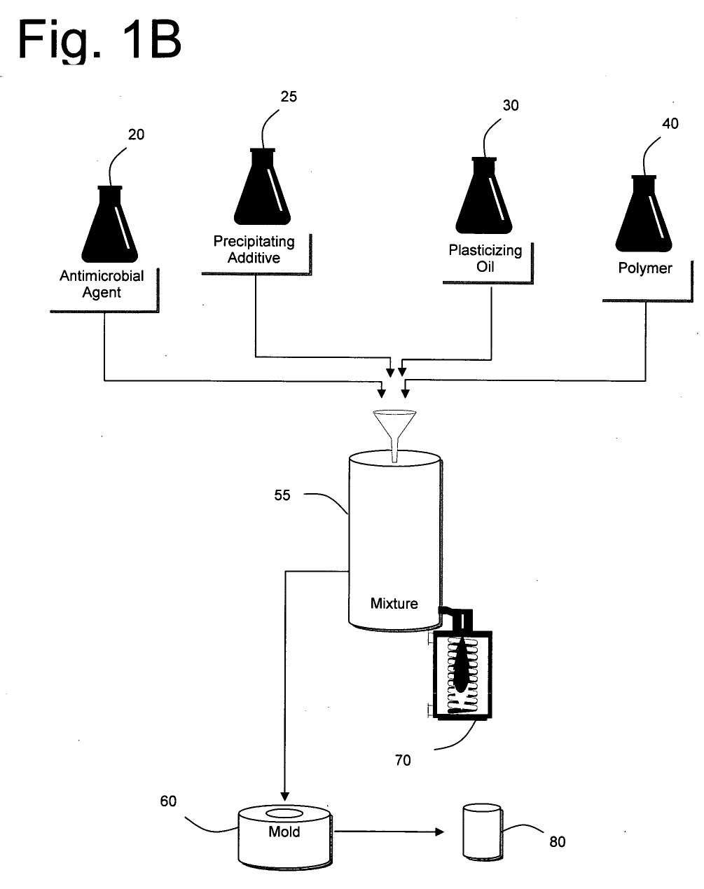

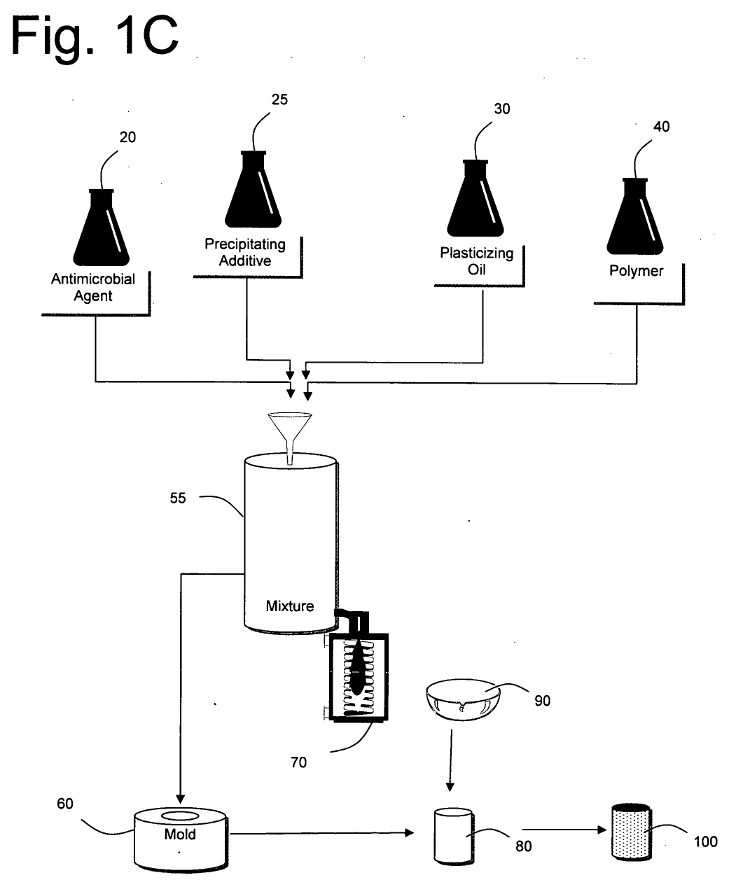

Turning to FIG. 1A, the novel process of making an elastomer includes the steps of antimicrobial agent 20, plasticizing oil 30, and polymer 40 to form mixture 50. Heat 70 is applied. Plasticizing oil 30 may be heated prior to the addition of antimicrobial agent 20 and polymer 40 thereto. Mixture 50 is melted in an extruder, a molding machine or other suitable heated vessel so that the antimicrobial agent 20 become suspended in molten mixture 50 and remain in stable suspension in the molten mixture. Molten mixture 50 is molded 60 into the form of a useful item to at an appropriate temperature. When allowed to cool, the mixture solidifies and forms elastomer 80. The antimicrobial agent 20 begins to diffuse to the surface of the elastomer upon completion of the solidification process.

If the plasticizing oil is heated, the appropriate temperature range is about 130 to 165° F. Plasticizing oils such as paraffinic oils, naphtenic petroleum oils, mineral oils, and synthetic liquid oilgomers of polybutene, polypropylene, polyterpene, etc. may be used. Preferably 300 to 1,200 parts by weight of the plasticizing oil may be used.

The inert nature and antimicrobial efficiency of silver make it an attractive option for the present invention. It is not toxic, flammable or corrosive and will not cause bacteria to become resistant to antibiotics. Silver stops bacteria or fungi degrading the object's physical properties, and also prevents the build-up of harmful bacteria, which can be a source of infection to humans. Microorganisms such as bacteria, fungi and algae can affect the aesthetic and physical properties of an elastomer by causing black spotting or discoloration, odor and polymer degradation. And in hospitals and care homes where patients are particularly vulnerable to infection, the build up of bacteria such as methicillin-resistant Staphylococcus aureus (MRSA) can contribute to the spread of deadly infections.

An advantage of silver-based additives is that they can be used in high temperature processing. For example, silver zirconium phosphate is thermally stable up to 800° C. The anti-microbial agents are mixed either in the dry polymer or in the mixture of polymer and plasticizing oil. Table 2 enumerates examples of suitable agents. Other compounds that provide silver ions are also suitable as well as antimicrobial agents.

TABLE 2

| | Chemical Name |
|---|---|
| 1 | Silver Zeolite |
| 2 | Silver Zirconium Phosphate |
| 3 | Silver Nitrate |
| 4 | Silver Thiosulfate |
| 5 | Silver Sulphadiazine |
| 6 | Silver Fusidate |
| 7 | Silver acetate |
| 8 | silver bromide |
| 9 | silver carbonate |
| 10 | silver chlorate |
| 11 | silver chloride |
| 12 | silver citrate |
| 13 | silver fluoride |
| 14 | silver iodate |
| 15 | silver lactate |
| 16 | silver nitrite |
| 17 | silver perchlorate |
| 18 | silver sulfide |

Silver-based antimicrobials use an ion exchange mechanism that slowly releases silver ions, which interact with the bonding sites on the microbe surface to prevent bacteria from reproducing. This slow, regulated release provides long-lasting effectiveness. In contrast, organic antimicrobials inhibit the growth of microbes by slowly leaching to the surface of the plastic, and subsequently into surrounding fluids. Such leaching can limit the durability of the additive and also cause discoloration and an unpleasant taste.

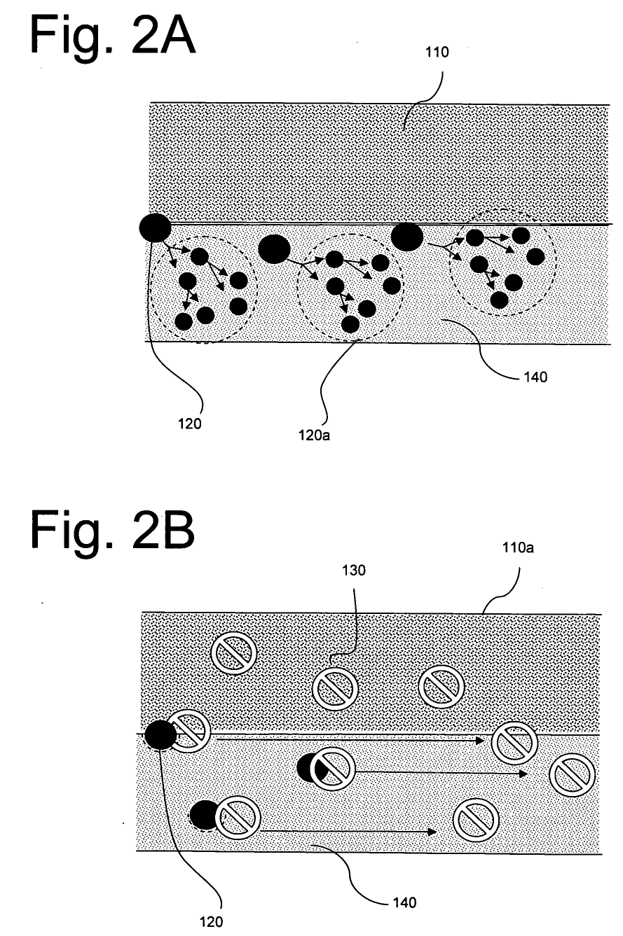

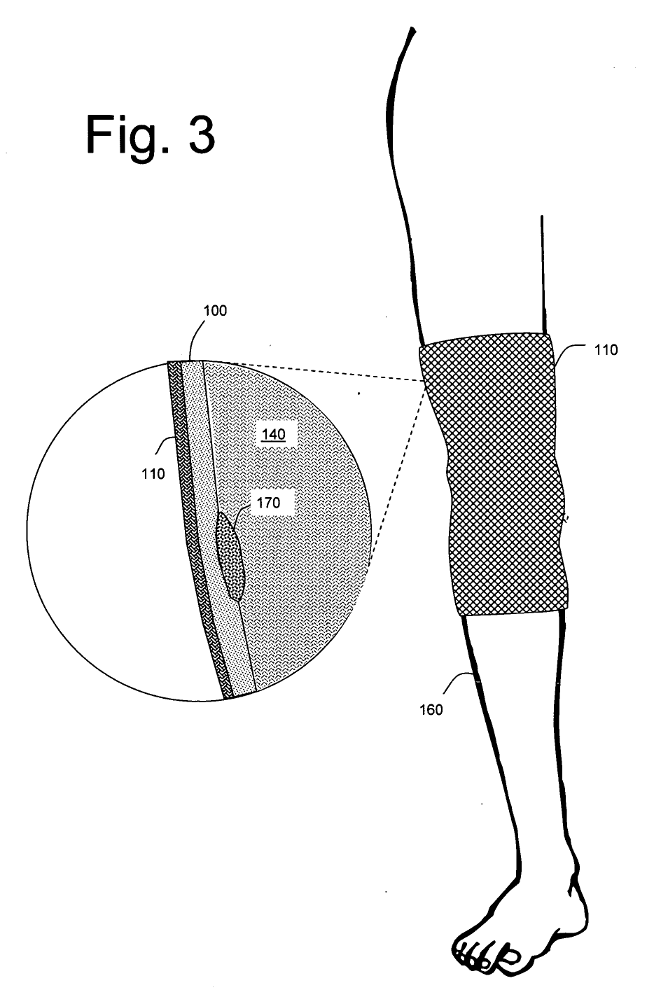

FIG. 2A illustrates the normal course of infection when conventional elastomers are used. As microbes 120 encounter a hospitable environment, such as skin 140, they begin to colonize. Microbes 120 begin to multiply and exponentially colonize the area 120a. In contrast, FIG. 2B shows that silver ions 130 slowly migrate from elastomer 110a toward tissue 140. The positive charge of ions 130 allows the silver ions to bond to the surface of microbes 120, thus interrupting reproduction. Since microbes 120 cannot reproduce, they eventually die and infection is thereby avoided. The silver ions are not consumed or dissolved in this process and therefore are able to continue their effectiveness.

The thermoplastic elastomer composition preferably comprises 100 parts by weight of triblock copolymer, 0.05 to 20 parts of one or more antimicrobial agent, and 100 to 900 parts of plasticizing oil The antimicrobial agent is solid at room temperature. The addition of such antimicrobial agent to the mixture of polymers and plasticizing oil is made either prior to the melting of the mixture in a heated vessel or when the mixture is in its molten state.

A polymer or mixture of polymers is added to the plasticizing oil for 30 minutes. The polymers may be of the type poly (styrene ethylene ethylene propylene styrene) (SEEPS), poly (styrene ethylene butylene styrene) (SEBS), or poly (styrene ethylene propylene styrene) (SEPS). These polymers are sold under the trademarks SEPTON and KRATON. Preferably, 100 parts by weight of one or a mixture of two or more of a hydrogenated styrene/isoprene/butadiene triblock copolymer are used.

The mixture containing the plasticizing oil, the antimicrobial agent and the polymer is melted in an extruder, a reciprocating screw molding machine, or a heated vessel at about 300 to 420° F. As mentioned earlier, the antimicrobial agent may be added to the mixture of polymers and plasticizing oils either prior to the melting of the mixture or in the melt phase.

In an alternate embodiment, FIG. 1B, an additive 25 is mixed with antimicrobial agent 20, plasticizing oil 30, and polymer 40 to form precipitating mixture 55. Precipitation mixture 55 is melted in an extruder, a molding machine or other suitable heated vessel so that the additives become soluble in precipitation mixture 55 and remain in stable solution in the molten mixture. Precipitating mixture 55 is molded 60 into the form of a useful item to at an appropriate temperature. When allowed to cool, the mixture solidifies and forms elastomer 80. The additives begin to diffuse to the surface of the elastomer upon completion of the solidification process. As shown in FIG. 1C, precipitation may be initiated by seeding the surface of elastomer 80 with fine powder 90 such as talcum powder. Elastomer 80 is cooled to solidified elastomer 100 whereby additive 20 precipitates to the surface of solidified elastomer 100 in the form of a dry powder.

Optionally, a seeding of the oil may also be effected, with an insoluble fine powder such as talc. Preferably, 300 to 1000 parts by weight of the plasticizing oil may be used. The additive is mixed in the plasticizing oil, optionally with seed, for approximately 10 minutes at 130 to 165° F. The additive may also be added to the plasticizing oil with or after the addition of the polymer. Table 3 discloses suitable additives.

TABLE 3

| | Chemical Name |
|---|---|
| 1 | Tetrakis (2,4-di-tert-butylphenyl) [1,1-biphenyl]-4,4′- |
| | diylbisphosphonite |
| 2 | Tris (2,4-ditert-butylphenyl) phosphate |
| 3 | Butanedioic acid, dimethylester, polymer with 4-hydroxy- |
| | 2,2,6,6-tetramethyl-1-piperidine ethanol |
| 4 | 2,6-di-tert-butyl-4-(4,6-bis(octylthio)-1,3,5-triazin-2-ylamino) phenol |
| 5 | 3,3′,3′,5,5′,5′-hexa-tert-butyl-a,a′,a′-(mesitylene-2,4,6-triyl) tri-p-cresol |
| 6 | Pentaerythritol Tetrakis (3-(3,5-di-tert-butyl-4- |
| | hydroxphenyl)propionate) |
| 7 | Phenol, 2-(5-chloro-2H-benzotriazole-2-yl)-6-(1,1-dimethylethyl)- |
| | 4-methyl |
| 8 | Thiodiethylene bis[3-(3,5-di-tert-butyl-4-hydroxyphenyl)propionate] |
| 9 | Calcium phosphonate |
| 10 | Dioctadecyl 3,3′-thiodipropionate |
| 11 | Didodecyl 3,3′-thiodipropionate |
| 12 | 2-(1,1-dimethylethyl)-6-[[3-(1,1-dimethylethyl)-2-hydroxy- |
| | 5-methylphenyl]methyl-4-methylphenyl acrylate |
| 13 | N,N′-hexane-1,6-diylbis(3-(3,5-di-tert-butyl- |
| | 4-hydroxyphenylpropionamide)) |

The tris (2,4-ditert-butylphenyl) phosphate as listed in Table I is a white crystalline powder, commonly used as a phosphate processing stabilizer for polycarbonate and polyolefins. It is typically used in combination with phenolic antioxidants and acts for synergistical color stability and polymer viscosity. The butanedionic acid as listed in Table I, also known as succinic acid, is a dicarboxylic acid with four carbon atoms, occurs naturally in plant and animal tissues and plays a significant role in intermediary metabolism (Krebs cycle). It is a colorless crystalline solid with a melting point of 185-187° C., soluble in water, slightly dissolved in ethanol, ether, acetone and glycerine, but not dissolved in benzene, carbon sulfide, carbon tetrachloride and oil ether. The common method of synthesis of succinic acid is the catalytic hydrogenation of maleic acid or its anhydride. Succinic acid has uses in certain drug compounds, in agricultural and food production, and in perfume esters.

The precipitate diffuses to the surface of the elastomer and collects as a powder on its surface. After removal of the surface powder, by wiping, washing, or the like, additional powder migrates to the surface. The process is repeated until the saturation level at room temperature of the precipitate in the elastomer is reached. The process of diffusion to the surface then stops.

Diffusion has several advantageous characteristics. The diffused precipitated phase modifies the surface characteristics of the elastomer by creating micro-craters on the elastomer surface.

A second advantage to the diffusion is that this process modifies the surface characteristics of the elastomer by providing a dry layer of microscopic powder. After an appropriate cooling period, the surface is powdery to the eye and to the touch. The surface modifications achieved by the novel method reduce the friction between the skin or other human tissue and the elastomer. The powdery molded reduces lateral movement from friction where the molded surface abuts epidermal tissue, for ecampl. Thus a lubricant may be added to the molded surface and retained by micro-craters prior to contact with epidermal tissue. This is an advantageous feature in applications such as burn patient treatment applications, scar reduction pads, wound care dressings, goggle frames, masks, headbands, orthotics, prosthetics, garments, urinary catheters, temporary implantations, and applications of cosmetics. Other applications not expressly mentioned herein are also within the scope of this invention as a matter of law.

The surface modifications are beneficial when the surface is wet with water or other liquid fluids. The micro-craters collect small pools of liquid which, in turn, provide additional lubricity. This is advantageous in medical, personal care, and cosmetic care applications, for example.

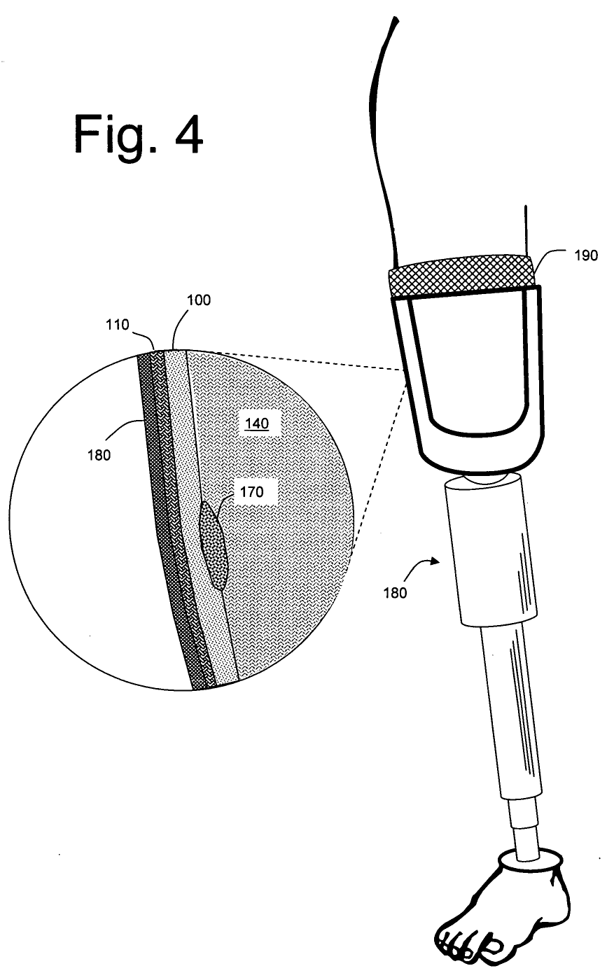

FIGS. 3-4 show an application of the present invention. In FIG. 3, wound 170 is located at the knee area of an individual's leg 160. Molded surface 110 of thermoplastic elastomer contacts wound 170 whereby antimicrobial agent 130 migrates from molded surface 110 to wound 170. In addition, the moisture impermeable properties of thermoplastic elastomer keep the wound area from drying out. Stretchable fabric 110 keeps a predetermined pressure of on the wound to control edema and/or other disorders relating to pressure on tissue and wounds.

FIG. 4 illustrates the same principle but with prosthetic socket 180 engaging leg 160 by liner 190 coated with thermoplastic elastomer on the inner surface of the fabric of the liner, in contact with wound 170. As thermoplastic elastomer has all the properties of a prosthetic liner an additional advantage of the present invention is to prevent, stem or cure infections caused by previously ill-fitted prosthetic devices. Wounds and subsequent infections may occur when a user is improperly fitted with a prosthetic device, the user improperly deploys the device or the user's body has changed since the prosthetic device was originally designed. The present invention may be used to treat and prevent further infections, reduce friction and stress on the tissue of a prosthesis wearer by incorporating the thermoplastic elastomer into a liner, sleeve or any other situation wherein a elastomeric surface must abut or compress against tissue.

A case where antimicrobial thermoplastic elastomer for prosthetic liners is of particular advantage is that of post-operative prosthetic liners, as the opportunity for serious infections is more likely immediately after surgery when the surgical sutures are still fresh.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1. An antimicrobial thermoplastic elastomer comprising:
a triblock co-polymer;
at least one plasticizing oil and a dispersion of at least one antimicrobial agent in the thermoplastic elastomer.

2. The thermoplastic elastomer of claim 1 further comprising at least one precipitating additive.

3. The thermoplastic elastomer of claim 2 wherein the precipitating additive is proportionally in excess of an amount of additive that is soluble in the elastomer at room temperature.

4. The thermoplastic elastomer of claim 1 further comprising a seeding of the oil with an insoluble fine powder mixed with the plasticizing oil.

5. The thermoplastic elastomer of claim 2 wherein the precipitating additive is selected from the group consisting of Tetrakis(2,4-di-tert-butylphenyl)[1,1-biphenyl]-4,4′-diylbisphosphonite; Tris(2,4-ditert-butylphenyl) phosphate; Butanedioic acid, dimethylester, polymer with 4-hydroxy-2,2,6,6-tetramethyl-1-piperidine ethanol; 2,6-di-tert-butyl-4-(4,6-bis(octylthio)-1,3,5-triazin-2-ylamino) phenol; 3,3′,3′,5,5′,5′-hexa-tert-butyl-a,a′,a′-(mesitylene-2,4,6-triyl)tri-p-cresol; and Pentaerythritol Tetrakis(3-(3,5-di-tert-butyl-4-hydroxphenyl)propionate).

6. The thermoplastic elastomer of claim 1 further comprising a precipitation seed on the elastomer.

7. The composition of claim 1 wherein the thermoplastic elastomer is impermeable to water.

8. The composition of claim 1 wherein the antimicrobial agent is silver-based.

9. The composition of claim 8 wherein the silver-based antimicrobial agent is selected from the group consisting of elemental silver, silver oxides, silver salts, silver ion exchange compounds, silver zeolites, silver glasses, and mixtures thereof.

10. The composition of claim 8 wherein the antimicrobial agent is selected from a group consisting of silver zeolite, silver zirconium phosphate, silver nitrate, silver thiosulfate, silver sulphadiazine, silver fusidate, silver acetate, silver bromide, silver carbonate, silver chlorate, silver chloride, silver citrate, silver fluoride, silver iodate, silver lactate, silver nitrite, silver perchlorate, and silver sulfide.

11. The composition of claim 1 wherein the triblock copolymer is selected from the group consisting of styrene-ethylene-ethylene-propylene-styrene, styrene-ethylene-butylene-styrene, and styrene-ethylene-propylene-styrene.

12. The composition of claim 1 where the plasticizing oil is 20% to 95% of the total composition.

13. An antimicrobial composition for use on human skin comprising:
a thermoplastic elastomer having a predetermined modulus of elasticity low enough to substantially reduce shear forces on skin ; and
a dispersion of silver-based antimicrobial agents in the thermoplastic elastomer whereby application of the thermoplastic elastomer over the skin permits the migration of the antimicrobial agents from the thermoplastic elastomer surface to the skin.

14. A method of making an antimicrobial wound or skin contact application comprising the steps of:
mixing a plasticizing oil and a polymer to form a mixture;
adding an antimicrobial agent to the mixture;
melting the mixture whereby the antimicrobial agents are suspended in the mixture; and
cooling the mixture forming a thermoplastic elastomer.

15. The method of claim 14 wherein the antimicrobial agent is silver-based.

16. The method of claim 15 wherein the silver-based antimicrobial agent is selected from the group consisting of elemental silver, silver oxides, silver salts, silver ion exchange compounds, silver zeolites, silver glasses, and mixtures thereof.

17. The method of claim 15 wherein the antimicrobial agent is selected from the group consisting of silver zeolite, silver zirconium phosphate, silver nitrate, silver thiosulfate, silver sulphadiazine, silver fusidate, silver acetate, silver bromide, silver carbonate, silver chlorate, silver chloride, silver citrate, silver fluoride, silver iodate, silver lactate, silver nitrite, silver perchlorate, and silver sulfide.

18. The method of claim 14 wherein the polymer is a triblock copolymer selected from the group consisting of styrene-ethylene-ethylene-propylene-styrene, styrene-ethylene-butylenes-styrene, and styrene-ethylene-propylene-styrene.

19. A method of making an antimicrobial wound application comprising the steps of: mixing together plasticizing oil and polymer to form a mixture; melting the mixture; adding an antimicrobial agent to the mixture whereby the antimicrobial agent is suspended in the melted mixture; and cooling the mixture until it solidifies forming a thermoplastic elastomer.

20. The method of claim 19 wherein the antimicrobial agent is silver-based.

21. The method of claim 20 wherein the silver-based antimicrobial agent is selected from the group consisting of elemental silver, silver oxides, silver salts, silver ion exchange compounds, silver zeolites, silver glasses, and mixtures thereof.

22. The method of claim 20 wherein the antimicrobial agent is selected from the group consisting of: silver zeolite, silver zirconium phosphate, silver nitrate, silver thiosulfate, silver sulphadiazine, silver fusidate, silver acetate, silver bromide, silver carbonate, silver chlorate, silver chloride, silver citrate, silver fluoride, silver iodate, silver lactate, silver nitrite, silver perchlorate, silver sulfide.

23. The method of claim 19 wherein the polymer is a triblock copolymer selected from the group consisting of styrene-ethylene-ethylene-propylene-styrene, styrene-ethylene-butylenes-styrene, and styrene-ethylene-propylene-styrene.

24. A dimensionally stable thermoplastic article comprising:
at least 51% of a thermoplastic elastomer; and
at least one silver-based antimicrobial agent.

25. The thermoplastic elastomer article of claim 24 wherein the thermoplastic elastomer is made of a triblock copolymer.

26. The thermoplastic article of claim 25 wherein the triblock copolymer is selected from the group consisting of Poly(Styrene-Ethylene-Butylene-Styrene), Poly (Styrene-Ethylene-Propylene-Styrene), and Poly (Styrene-Ethylene-Ethylene-Propylene-Styrene).

27. The thermoplastic article of claim 24 further comprising a plasticizing oil.

28. The thermoplastic article of claim 27 wherein the plasticizing oil is selected from the group consiting of paraffinic, naphtenic, synthetic liquid oligomers of polybutene, polypropene, and polyterpene.

29. The thermoplastic elastomer article of claim 24 comprising at least one silver ion control release additive.

30. The elastomer article of claim 29 wherein the at least one silver ion control release additive is selected from the group consisting of fillers, oils, pigments, salts, antistatic agents, and mixtures thereof.

31. The thermoplastic elastomer article of claim 24 wherein the silver-based antimicrobial agent is selected from the group consisting of elemental silver, silver oxides, silver salts, silver ion exchange compounds, silver zeolites, silver glasses, and mixtures thereof.

32. The thermoplastic elastomer article of claim 24 wherein the antimicrobial agent is selected from a group consisting of silver zeolite, silver zirconium phosphate, silver nitrate, silver thiosulfate, silver sulphadiazine, silver fusidate, silver acetate, silver bromide, silver carbonate, silver chlorate, silver chloride, silver citrate, silver fluoride, silver iodate, silver lactate, silver nitrite, silver perchlorate, and silver sulfide.

33. The thermoplastic article of claim 24 wherein the article is chosen from the group consisting of prosthetic liners, shoe insoles, medical appliances designed for contact with human skin, CPAP (continuous positive air pressure) masks, external breast prosthesis, breast augmentation pad for insertion into brasseries, prosthetic suspension sleeves, appliances for personal care designed for contact with human skin, metatarsal pads, wound dressing sheets, wound dressing pads, catheters for vascular use, catheters for urinary use, inflation balloons for medical catheters, and malleolus pads.
